(19) United States
(12) Patent Application Publication
(10) Pub. No.: US 2016/0010142 A1
(43) Pub. Date: Jan. 14, 2016

(54) Compositions and Methods For Analysis of C02 Absorption

(71) Applicant: NOVOZYMES NORTH AMERICA, INC., Franklinton, NC (US)

(72) Inventors: Sonja Salmon, Raleigh, NC (US); Alan House, Cary, NC (US); Margaret Whitener, Belmont, NC (US)

(73) Assignee: NOVOZYMES NORTH AMERICA, INC., Franklinton, NC (US)

(21) Appl. No.: 14/769,520

(22) PCT Filed: Mar. 14, 2014
(86) PCT No.: PCT/US2014/028597

§ 371 Date: Aug. 21, 2015

Related U.S. Application Data
(60) Provisional application No. 61/788,562, filed on Mar. 15, 2013.

(51) Int. Cl. C12Q 1/527

(52) U.S. Cl. CPC C12Q 1/527; G01N 2333/988

(57) ABSTRACT

The present invention relates to methods for analysis of carbon dioxide (CO2) absorption into a liquid enhanced by the presence of a compound that accelerates the absorption reaction resulting in a more rapid pH change in the liquid than in the absence of the compound. The present invention also relates to catalyst solutions comprising a carbonic anhydrase and a buffer effective in enhancing CO2 absorption. The present invention further relates to improved compositions and methods for carbon dioxide (CO2) absorption using a zinc additive.

### CROSS-REFERENCE TO RELATED APPLICATIONS

This application claims priority from U.S. provisional application Ser. No. 61/788,562 filed Mar. 15, 2013, the content of which is fully incorporated herein by reference.

### STATEMENT OF GOVERNMENT INTEREST

This invention was made with Government support under Prime Contract No. DE-FE0007741 awarded by the Department of Energy. The Government has certain rights in this invention.

### FIELD OF THE INVENTION

The present invention relates to methods for analysis of carbon dioxide (CO₂) absorption into a liquid enhanced by the presence of a compound that accelerates the absorption reaction resulting in a more rapid pH change in the liquid than in the absence of the compound. The present invention also relates to catalyst solutions comprising a carbonic anhydrase and a buffer effective in enhancing CO₂ absorption. The present invention further relates to improved compositions and methods for carbon dioxide (CO₂) absorption using a zinc additive.

### BACKGROUND OF THE INVENTION

Carbon dioxide (CO₂) emissions are a major contributor to the phenomenon of global warming. CO₂ is a by-product of combustion and it creates operational, economic, and environmental problems. CO₂ emissions may be controlled by capturing CO₂ gas before emitted into the atmosphere. There are several chemical approaches to control CO₂ emissions. One approach is to pass the CO₂ through an aqueous liquid containing calcium ions, allowing the CO₂ to precipitate as CaCO₃. Preferred techniques for capturing CO₂ gas from combustion processes are ones in which the product of the capture process is CO₂ in the form of a gas that can be compressed and transported to storage sites, or for useful purposes.

The most well-established technique for extracting CO₂ from a gaseous feed and capturing the extracted CO₂ gas for use or storage is absorption of CO₂ into aqueous solutions, such as aqueous solutions of amines, for example, monoethanolamine (MEA) or methyldiethanolamine (MDEA), or aqueous solutions of inorganic salts, such as potassium carbonate, sodium carbonate, ammonium carbonate, potassium phosphate, or sodium phosphate. In the case of primary amines, such as MEA, CO₂ is mainly described as being absorbed as a result of chemical reaction between CO₂ and the amine to form a carbamate compound. Additional absorption can occur as a result of ionic complexation between protonated amines and CO₂ molecules which have been hydrated to bicarbonate. In the case of tertiary amines, such as MDEA, absorption occurs as a result of ionic complexation between protonated amines and CO₂ molecules which have been hydrated to bicarbonate. In the case of inorganic salts, absorption occurs as a result of ionic complexation between the cation of the salt, such as potassium or sodium, and CO₂ molecules which have been hydrated to bicarbonate. CO₂ hydration preferentially occurs at alkaline pH and results in a decrease in pH as the conversion of CO₂ to bicarbonate increases. Depending on the pH, an equilibrium of carbonic acid, bicarbonate and carbonate ions will be present in the solution. To complete the CO₂ capture process and recycle the solvent, after absorption, a driving force, such as heat and/or a change in pressure, is typically used in one or more desorption process stages to release CO₂ from the “CO₂—Rich” absorption solution and recycle the “CO₂-Lean” absorption solution back to the absorption stage. Alternatively, CO₂-containing anions in the absorption solution may be precipitated as insoluble salts, such as calcium carbonate, and removed from the liquid in the solid form.

Catalysts are able to increase the rate of the reversible CO₂ hydration (forward reaction) and dehydration (reverse reaction) reactions shown in the following reaction.

CO₂+H₂O [symbol] H⁺+HCO₃⁻

Certain biological catalysts, such as the enzyme carbonic anhydrase, can catalyze the conversion of CO₂ to bicarbonate at a very high rate (turnover numbers up to 10⁶ molecules of CO₂ per second are reported).

Selection of a high performing carbonic anhydrase to catalyze the CO₂ hydration reaction under application relevant conditions depends on several factors including: (i) enzyme robustness to temperature stress or impurities or enzyme poisons that may be present, (ii) enzyme longevity and (iii) enzyme activity. The degree to which an enzyme resists temperature stress or impurities or enzyme poisons, the length of time an enzyme retains activity when exposed to certain conditions, and the magnitude of a particular enzyme sample's activity can all be assessed using an enzyme activity assay, such as provided by embodiments of the present invention. Selection of a high-performing non-enzyme catalyst, or a combination of enzyme and non-enzyme catalysts, also depends on the factors described above. Examples of non-enzyme catalysts that can be evaluated according to the methods of the present invention are zinc-centered organo-metallic compounds, such as zinc-centered tris(triazolyl)pentaerythritol and analogous compounds, which are reported to catalyze the hydration of carbon dioxide (U.S. Pat. No. 8,394,351).

Several assays for carbonic anhydrase activity have been described in the literature. These assays involve diluting the enzyme in aqueous buffers to either assess enzyme esterase activity by measuring p-nitrophenyl acetate conversion to p-nitrophenol (for example, Bond, G. M. et al. 2001. Energy and Fuels. 15: 309); or assess enzyme CO₂ hydration/HCO₃⁻ dehydration activity by measuring the change in pH that accompanies each reaction, either by using a pH meter or a pH sensitive colorimetric indicator (for example, Wilbur, K. M. and N. G. Anderson. 1948. J.

Biol. Chem. 176: 147), or by measuring the change in gas pressure in the headspace over the reaction by using manometry (for example, Roughton, F. J. W. and V. H. Booth. 1946. Biochem. J. 40: 309).

Assays for CO₂ hydration typically use CO₂ saturated water as substrate, converting CO_{2(g)} (carbon dioxide molecules in the gas phase) to CO_{2(aq)} (carbon dioxide molecules dissolved in aqueous solution). Using CO₂ saturated water divorces the rate of gas-liquid mass transfer from the enzyme catalyzed rate of CO_{2(aq)} hydration, and allows the assay to only measure the hydration activity. Alternatively, assays for CO₂ hydration can involve exposure of the assay liquid to a headspace containing CO₂ gas. In this case, the reaction rate of the assay will be influenced by both the rate at which CO₂ gas encounters and enters the liquid phase, and the rate at which dissolved CO₂ is converted to bicarbonate. The partial pressure of CO₂ in the headspace will influence the reaction rate. A high partial pressure of CO₂ in the headspace will typically accelerate the absorption reaction. A low partial pressure of CO₂ in the headspace will typically result in a slower absorption reaction. A very low partial pressure of CO₂ in the headspace, such as could be achieved by sweeping the headspace with a non-CO₂ gas, such as nitrogen gas, or applying a vacuum to the headspace will typically result in the rate of the desorption reaction occurring faster than the rate of absorption. An example of a method that utilizes the presence and absence of CO₂ in the headspace to carry out the dehydration reaction for monitoring carbonic anhydrase activity has been reported (WO 2010/081007). Aliquots of assay reaction mixture containing aqueous potassium carbonate and phenolphthalein pH indicator dye and different levels of carbonic anhydrase were first exposed to a 20% CO₂ atmosphere, which caused the solution pH to decrease and the pH indicator to turn colorless. The colorless aliquots were removed from the 20% CO₂ atmosphere and the bicarbonate dehydration rate was determined by monitoring the change in absorbance at 550 nm using a plate reader. Carbonic anhydrase activity was calculated from the onset time at which the absorbance of the reaction mixture reached a set optical density value.

Although published methods can be used, there is no uniform standard, indicating the established methods have drawbacks and there is a continued need for improved methods for analysis of enhanced CO₂ absorption. There is also a need for improved carbonic anhydrase compositions for use in CO₂ absorption.

### SUMMARY OF THE INVENTION

Facilitation of CO₂ absorption and desorption using catalysts, such as carbonic anhydrase, is important for applications requiring separation of CO₂ from mixed gases, such as, CO₂-containing gases such as flue gas from power plants burning fossil fuel (e.g. coal or natural gas) or biomass (e.g. wood) or combustible waste materials (e.g. municipal waste) or mixtures of these, biogas, landfill gas, ambient air, recycled air (such as in a confined environment), synthetic gas or natural gas or any industrial or process off-gas containing carbon dioxide, and/or transformation, mineralization and/or utilization of CO₂ in the hydrated bicarbonate form, such as for enhanced algae growth and pH control or adjustment. Improved CO₂ absorption and desorption analytical methods are needed to reduce variability in the analyses, simplify the analyses, make the analyses more application relevant, such as by conducting the analyses at or above ambient temperature, increase sample throughput, and decrease the use of reagents. Improved analytical methods can be used to efficiently identify new catalysts and monitor the performance of catalysts.

The present invention relates to improved methods for analysis of carbon dioxide (CO₂) absorption and desorption into a liquid enhanced by the presence of a compound that accelerates the absorption reaction resulting in a more rapid pH change in the liquid than in the absence of the compound. The compound may react chemically with CO₂ to form a new compound while concurrently causing a pH change, or, in some embodiments, the compound is a catalyst, such as an enzyme catalyst, that accelerates CO₂ absorption without forming a sustained chemical bond with CO₂. In some embodiments, the enzyme catalyst is carbonic anhydrase.

The improved methods additionally relate to novel combinations of aqueous solutions and indicators that improve CO₂ absorption assay reliability due to having similar acid dissociation constants (pKa). Preferably, the improved methods comprise aqueous solutions of a buffer compound comprising a tertiary amine together with an indicator, wherein the buffer compound and the indicator have similar acid dissociation constants (pKa), wherein the aqueous solution is used in an assay measuring CO₂ absorption and desorption. Preferably, the improved methods comprise aqueous solutions of bicine together with cresol red, a pH indicator. The methods utilize carbon dioxide (CO₂) as the assay substrate and measure the ability of a compound, such as carbonic anhydrase, to accelerate CO₂ absorption by aqueous solutions for useful applications. Results of performing the methods can be presented as a numerical quantification of the enzyme activity, useful, for example, for allowing numerical comparison of the catalytic activities among different samples.

The improved methods also relate to analysis of CO₂ desorption out of aqueous liquids containing bicarbonate and/or carbonic acid, enhanced by the presence of a compound that accelerates the desorption reaction resulting in a more rapid pH change in the liquid than in the absence of the compound. In some embodiments, the compound is a catalyst, such as an enzyme catalyst, that accelerates CO₂ desorption without forming a sustained chemical bond with bicarbonate, carbonic acid or CO₂. In some embodiments, heat or vacuum or a combination of these is applied during the method to exaggerate the desorption effect.

Measurement of the performance of non-catalyst compounds that influence CO₂-hydration and/or dehydration reactions can also be made using the methods of the present invention, which is useful for the selection or identification of such compounds, for example, surfactants or salts. In one embodiment of the invention, the effect of compounds that inhibit the catalytic performance of a CO₂-hydration and/or dehydration enhancing compound can be measured by methods of the present invention.

The present invention relates to improved compositions for CO₂ absorption comprising an aqueous solution comprising carbonic anhydrase and a buffer compound comprising a tertiary amine, wherein the buffer compound is added in an amount effective to enhance CO₂ absorption as a result of the carbonic anhydrase activity. The present invention relates to improved compositions for CO₂ absorption comprising an aqueous solution comprising bicine, wherein bicine is added in an amount effective to enhance CO₂ absorption as a result of the carbonic anhydrase activity.

The present invention also provides methods for improving the activity of a carbonic anhydrase comprising adding zinc to a composition comprising one or more carbonic anhydrases. Such methods may be applied in the context of a method for carbon dioxide (CO₂) absorption. The invention also relates to compositions comprising one or more carbonic anhydrases and zinc ions, wherein the zinc ions were added to the composition independent of the natural zinc content of the one or more carbonic anhydrases. The zinc ions are added in an amount effective to increase the catalytic activity of a carbonic anhydrase.

### DRAWINGS

FIG. 1 shows a graph of enzyme activity (change in absorbance with change in time) as a function of enzyme solution volume measured using an embodiment of the assay methods of the present invention.

FIG. 2 shows a graph comparing the assay measurement using different embodiments of the assay methods of the present invention.

FIG. 3 shows data collected using the assay methods of the present invention to determine the effect of salt concentration on the determination of carbonic anhydrase activity for a form of the enzyme that precipitates in the absence of salt.

FIG. 4 shows data collected using a bubble tank reactor and used to graph the percent of CO₂ absorbed over time in solutions comprising mixtures of bicine, carbonate and carbonic anhydrase.

### DEFINITIONS

The “aqueous CO₂ substrate” is a solution of (e.g., deionized) water that has been saturated with CO₂ gas, typically by exposing the water to partial pressures of CO₂ gas that are above the ambient partial pressure of CO₂ gas in the environment where the assay is conducted, causing CO₂ gas molecules to dissolve in the water phase. The abbreviation CO_{2(aq)} is used to describe CO₂ gas molecules dissolved in the water phase. In some embodiments, deionized water is used to make the CO₂ gas saturated solution. In other embodiments, CO₂ in the form of dry ice is used to saturate water. Optionally the “assay substrate” is equilibrated to the temperature of the assay prior to use.

The term “assay blank” is used in the present invention to refer to the combination of “blank” suitably diluted in “assay reagent”, or when allowed by test criteria refers to “assay reagent” alone.

The term “assay buffer” is used in the present invention to describe an aqueous solution of a chemical compound, or mixture of compounds, that will hold an approximately constant pH de-spite fluctuations in hydrogen ion concentration through a defined pH range that is typically one pH unit above or below the pKa of the compound or mixture of compounds.

The term “assay liquid” is used in the present invention to refer generically to either an “assay sample” or an “assay blank.”

The term “assay reagent” is used in the present invention to refer to the combination of “assay buffer” together with “pH indicator” in aqueous solution.

The term “assay sample” is used in the present invention to refer to the combination of “test sample” suitably diluted in “assay reagent.”

The term “assay substrate” is used in the present invention to refer generically to either an aqueous CO₂ substrate (when measuring the hydration reaction) or an aqueous bicarbonate substrate (when measuring the dehydration reaction), such as, a solution of bicarbonate salt (for example NaHCO₃ or KHCO₃) diluted in (e.g., deionized) water to achieve a target initial bicarbonate concentration.

The term “blank” is used in the present invention to refer to a liquid that gives a response in the assay equivalent to the response of a “test sample” minus the compound(s) causing enhanced CO₂ absorption or desorption, or a liquid in which the compound(s) causing enhanced CO₂ absorption or desorption have been inactivated. Test criteria for selecting suitable blanks are provided such that if no difference in assay response is measured between these “blanks” relative to the “assay reagent” alone, then “assay reagent” alone is the preferred composition of the “blank.”

The terms “CO₂-lean” and “CO₂-rich” liquids are terms used in the present invention to describe the relative amount of carbon (in the form of dissolved CO₂, chemically reacted CO₂, bicarbonate, carbonic acid and/or carbonate salt) present in the liquid. As used herein, the term “CO₂-lean liquid” generally refers to liquid that is capable of absorbing CO₂, such as a liquid that would absorb CO₂ from a CO₂-containing gas in the absorption stage of a CO₂ removal process. The term “CO₂-rich liquid” generally refers to a liquid that is capable of releasing CO₂, such as a liquid that would release CO₂ from a CO₂-containing liquid in the desorption stage of a CO₂ removal process. It is understood that the term “CO₂-lean liquid” can also be applied to liquid exiting or at the completion of a desorption stage, and the term “CO₂-rich liquid” can also be applied to liquid exiting or at the completion of an absorption stage.

The term “pH indicator” is used in the present invention to describe a chemical compound that imparts a pH dependent color to an aqueous solution. The color of the solution is dictated by the equilibrium between acidic and basic forms of the compound such that a change in hydrogen ion concentration causes a shift in equilibrium and a consequent shift in color.

The term “test sample” is used in the present invention to refer to an undiluted sample to be evaluated using analysis methods of the invention.

### DETAILED DESCRIPTION OF THE INVENTION

The present invention describes the use of a buffer compound in an assay for measuring CO₂ absorption and desorption in combination with a pH indicator. Typical assays take advantage of the change in pH that accompanies the CO₂ hydration or dehydration reaction depicted in the following reaction carried out under buffered conditions:

CO₂+H₂O [symbol] H⁺+HCO₃⁻

A pH sensitive colorimetric indicator is employed in the assay to analyze catalysis activity (e.g. carbonic anhydrase activity) using either dissolved CO₂ or bicarbonate as substrates for the hydration or dehydration reactions, respectively. Accordingly, the reaction is followed in a buffered solution such that in addition to enzyme and substrate the buffered aqueous solution contains a buffer-indicator pair.

The present invention particularly describes the use of a buffer compound in combination with a color indicator having the same or similar acid dissociation constants (pKa). Moreover, in one embodiment, for analysis of the CO₂ adsorption reaction, it is advantageous for the color transition point of the indicator to occur outside of the buffer range of the assay to ensure a sharp and well defined final color transition point. Accordingly, in an embodiment, the present invention describes the use of a buffer compound with an indicator that has the following characteristics: (i) having a pKa which is similar to the pKa of the buffer compound; and (ii) which enables a color transition point which occurs outside of the buffer range controlled by the buffer compound.

As used herein the phrase “similar” in referring to different pKa values means that the pKa of one compound (e.g., buffer compound) differs from the pKa of the other compound (indicator) by no more than 0.5 unit, such as, no more than 0.4 units, no more than 0.3 units, no more than 0.2 units, no more than 0.1 units, no more than 0.09 units, no more than 0.08 units, no more than 0.07 units, no more than 0.06 units, no more than 0.05 units, no more than 0.04 units, and no more than 0.03 units.

A suitable buffer compound of the present invention forms an aqueous solution that has a pH falling in the preferred ranges. In some embodiments, the buffer compound for use in the invention is an N, N-disubstituted derivative of an amino acid which comprises a tertiary amine functional group. Primary and secondary amine functional groups are known to react covalently with CO₂ to form carbamates which can interfere in a CO₂ hydration assay, accordingly, in some embodiments, the buffer compound also does not contain a primary or secondary amine functional group.

An example of a suitable buffer compound for use in the assay is bicine, which is also known as 2-(Bis(2-hydroxyethyl)amino)acetic acid, N,N-Bis(2-hydroxyethyl)glycine, diethylolglycine, Diethanol glycine, Dihydroxyethylglycine, and DHEG; CAS Number 150-25-4. An advantage of bicine as a buffer for CO₂ absorption analysis is that bicine contains a tertiary amine which will not react covalently with CO₂ to form compounds that could interfere with the CO₂ absorption reaction. Bicine is an N,N-disubstituted derivative of the amino acid glycine. The di-N-hydroxyethyl derivatization alters the pKa of glycine to a pKa suitable for the methods of the present invention. Other suitable buffers are N,N-disubstituted derivatives of other amino acids (e.g., alanine, leucine or isoleucine) providing a pKa similar to that of bicine. In some embodiments, the N,N-disubstituted derivative comprises at least hydroxy-substituted alkanyl moiety, e.g., a hydroxymethyl, hydroxyethyl, hydroxylpropyl, and/or hydroxylisopropyl moiety. Suitable derivatives of amino acids may, in some examples, have a 2-hydroxyethyl group as one or two of the N,N-disubstituted chemical groups.

The most suitable concentration of buffer compound should be optimized for the reaction assay, since it is dependent on several parameters such as CO₂ concentration provided as the substrate, catalyst concentration (e.g., carbonic anhydrase), and temperature. In one embodiment of the present invention, the buffer compound is bicine. A suitable concentration of buffer compound (e.g., bicine) is, e.g., between 1 mM and 2 M, such as between 5 mM and 1.5 M, between 10 mM and 1 M, or between 10 mM and 100 mM. In some embodiments, the initial pH of the buffer is maintained above pH 7.5, e.g., the initial pH is maintained between 8 and 10, such as between 8 and 9, between pH 8.0 and 8.5, or between 8.3 and 8.7. In some embodiments, the buffer compound in the assay liquid is bicine and the assay buffer is adjusted to pH about 8.3, or pH between pH 8.3 and 8.7 prior to use in the assay.

An example of a suitable indicator is cresol red, a triarylmethane dye, which is also known as o-Cresolsulfonephthalein; CAS Number 1733-12-6. Cresol red is particularly suitable for use in combination with the buffer compound bicine. The buffer range for bicine is pH 7.4-9.3. Bicine (pKa 8.35) and cresol red (pKa 8.32) have similar pKa values that differ by only 0.03 units. However, the color transition point for cresol red occurs at pH 7.0, which is outside the bicine buffer range. At pH values below the color transition point, cresol red provides a visually yellow color in aqueous solution. At pH values above the color transition point, cresol red provides a gradient of visually orange to red colors in aqueous solution, depending on the pH. At pH values above approximately pH 9, the visual color of cresol red in aqueous solution is red to purple, depending on the pH. A typical human observer can readily detect the color change that occurs when the red-orange color of cresol red changes to yellow as pH of the aqueous liquid changes across the pH of the color transition point. Analytical instruments that measure color, e.g. by measuring absorbance of certain wavelengths of light, can also be used to detect the described color changes. As previously indicated, having a sharp color transition point is especially useful, for example, when determining the time required to reach a reaction end-point, where a sharp and well defined color transition increases reliability of the endpoint measurement.

Another advantage of cresol red as an indicator (such as, in combination with bicine) is that cresol red has a relatively high water solubility compared to other indicators with similar pKas, such as phenol red. Higher water solubility results in easier solution preparation and use, such as the ability to prepare concentrated stock solutions that can be easily measured, combined and/or diluted with other assay components, such as saturated CO₂ water, to achieve the necessary dilutions and/or final reaction volume. Moreover, increased indicator solubility promotes efficient mixing of the assay liquid and assay substrate following assay substrate addition prior to data collection. Because catalyst kinetics can be fast, the time between assay substrate addition and data collection can be short. Thus more efficient mixing can lead to a homogenous assay liquid plus assay substrate solution in a shorter amount of time and thereby improve data quality.

Accordingly, in an embodiment, the method comprises the use of bicine and cresol red in a method for analysis of enhanced CO₂ absorption. In a one embodiment, the method is used for analysis of carbonic anhydrase enzyme activity.

Assays are typically conducted at slight alkaline pH (approx. pH 8) to mitigate the effect of hydroxide ion mediated CO₂ conversion to bicarbonate (HCO₃—) at around pH>10, and the reaction between CO₂ and H₂O to yield carbonic acid (H₂CO₃) at around pH<6. Including a buffering agent(s) in the assay liquid is important to both facilitate solution preparation in order to achieve the target initial pH and to slow the rate of pH change that results from the change in hydrogen ion concentration as the reaction proceeds (as depicted in the above reaction). A slower rate of reaction can facilitate measurement of pH change within the target assay pH range (pH 6-10). Furthermore, without being bound by any specific mechanism, a buffering agent(s) can facilitate proton transfer from the enzyme active site, which may be a rate limiting step in the absence of a buffering agent(s) (Silverman, D. N. and C. K. Tu. 1975. J. Am. Chem. Soc. 97:2263). In alkaline solutions, in the absence of added catalyst, CO₂ hydration occurs as a result of the chemical reaction between CO₂ and hydroxide anion.

The measure of catalyst activity can be either the time required to achieve an endpoint pH (or endpoint absorbance or color when employing a pH sensitive colorimetric indicator), or the slope of the change in pH (or absorbance) per unit time to give the reaction rate. When the purpose of the analysis is to determine the reaction rate, it is important to ensure that the catalyst is saturated with substrate; not only as a principle of catalyst activity rate determinations, but also to ensure reproducibility of the assay from one experiment to the next, for example, in order to compare catalyst longevity or robustness to different temperature stresses or impurities or poisons. In these cases, it is important that catalyst active sites are saturated with substrate at all times to ensure a decline in catalyst performance can be differentiated from simply having fewer catalyst molecules colliding with substrate due to substrate limitations. Observation of a linear rate (change in pH per unit time) can be used to determine whether the catalyst is saturated with substrate. When using a pH sensitive colorimetric indicator, it is important to closely match the pKa of the buffer and indicator to ensure the indicator is a true measure of pH, providing a linear response that directly corresponds to the CO₂ hydration reaction. When the pKa of the assay buffer and pH indicator are not closely matched, such as is the case with certain methods presented in the literature (Sodium carbonate/sodium bicarbonate buffer with phenol red indicator, pKa=10.3 and 8.0, respectively (Maren, T. H. et al. 1960. J. Pharmacol. Exp. Ther. 130: 389); and Veronal buffer with bromothymol blue indicator, pKa=8.0 and 7.1, respectively (Wilbur, K. M. and N. G. Anderson. 1948. J. Biol. Chem. 176: 147)), the likelihood of non-linear assay responses is increased, resulting in higher variability of the assay results, and decreased ability to rely on the assay to detect differences between test samples.

### Data Processing

In a one embodiment of this invention, a data processing template created using a spreadsheet program that provides statistical calculation tools (e.g., Microsoft Excel) is used to permit the rapid evaluation of high-throughput analysis data collected using a microtiter-plate reader. The data processing template provides a mathematical determination of whether or not the catalyst, such as an enzyme catalyst, is saturated with substrate during the activity measurement. As long as the enzyme is saturated with substrate it can be expected to generate protons at a constant velocity. In the constant velocity response range, the change in proton concentration between data points should be linear, translating to a linear change in absorbance versus time. As enzyme concentration increases, the velocity will also increase, and will remain linear for as long the enzyme remains saturated with substrate. As soon as the reaction becomes substrate-limited, the velocity will no longer be linear. Data processing rules incorporated in the template ensure linearity of accepted measurements. A set of serial dilutions of the test sample is performed in order to ensure active enzyme is saturated with substrate during the analysis. The data processing template informs whether or not an appropriate dilution range has been used in generating results from a particular data set.

Well known statistical tests are the “F-test” and “T-test” used to determine the acceptability of a particular data set. An F-test between assay blank and assay sample replicate slopes may be performed to determine whether the variances between the data sets are significantly different dictating whether a homoscedastic or heteroscedastic T-test should be performed. A T-test between assay blank and assay sample replicates may be performed to determine the probability that the differences between the two data sets could arise by chance, and ensures that the test sample has not been excessively diluted in preparing the assay sample. The number of replicates passing the linearity and T-tests are considered in determining whether or not to include the data in the final activity determination.

Thus, elements of the data processing template include a linear ratio measurement to compare the slopes between data points among a multi-data point slope within the assay window. A linear ratio of 1 translates to a perfectly linear slope, which corresponds to a perfectly constant initial velocity within the linear velocity test window. Linear ratios that fall outside the acceptable range are deemed not linear and rejected. In one embodiment of the invention, the acceptable range for the linear ratio is 0.8 to 1.2. In one embodiment of the invention, the acceptable range for the linear ratio is 0.85 to 1.18. In another embodiment of the invention, the acceptable range for the linear ratio is 0.9 to 1.1. The determined linear slopes for the assay blank reaction is subtracted from the assay sample to determine the rate contribution of the enzyme at that enzyme concentration.

Final activity is determined from the slope of measured activities versus volume of test sample in the assay sample and may be reported as units per unit volume (for example, milliliters). Alternatively, appropriate conversion factors can be used to express the activity in other terms, such as units per mass (for example, milligrams) enzyme protein, when the protein concentration of the liquid test sample is known (or the enzyme protein content by mass of, for example, a solid test sample, is known).

Calculating kcat.

In one embodiment of the invention the catalytic constant (kcat) of carbonic anhydrase can be calculated using the assays provided herein. The catalytic constant is defined as “the number of moles of substrate converted to product under saturating conditions per second per mole of enzyme [active site]” (Horton, R. H. et al. 1996. Principles of Biochemistry. 2nd ed. Prentice-Hall Inc., Upper Saddle River, N.J.). Because enzyme saturation is a requirement for data inclusion during data processing, each constant velocity measurement included represents the maximum velocity (Vₘₐₓ) for a particular enzyme concentration, thereby satisfying the requirements for determining kcat:

k_{cat}=Vₘₐₓ/([Enzyme]) Equation 1.

In order to convert between a velocity in terms of ΔAbsorbance/Δtime to one of ΔCO₂ hydration/Δtime, a calibration curve is created by plotting absorbance as a function of moles of protons added to the assay reagent Thus, the starting solution would contain no moles of protons added. As successive moles of protons are added, the colorimetric change will be recorded and the calibration plot can be used to convert ΔAbsorbance/Δtime to Δ[H+]/Δtime. Since one mole of H+ added corresponds to one mole of CO₂ hydrated (according to the above reaction), we can equate the Δ[H+]/Δtime with ΔCO₂ hydration/Δtime (as discussed in Khalifah, R. G. 1971. J. Biol. Chem. 246: 2561). Therefore, a k_{cat} corresponding to moles CO₂ hydrated per second per mole enzyme can be calculated.

For practical reasons, the enzyme activity unit of ΔAbsorbance/Δtime may be more useful than the kcat value for routine screening because slight changes in indicator (e.g., cresol red) concentration among solution preparation batches may affect the calibration curve, such that a separate calibration curve is required for each batch.

### Equipment and Processes

One aspect of the invention is a multi-well microplate reader capable of carrying out absorbance measurements in the visible spectrum and, preferably, equipped with a liquid dosing system that can be programmed to automatically dispense a specific volume of aqueous CO₂ substrate individually to the assay liquid in each reaction vessel, such as to each well of a 96-well plate. Equipment useful for the methods of the present invention are available commercially, for example the Infinite® M1000 model microplate reader equipped with liquid dispensing (TECAN Group Ltd., Seestrasse 103, CH-8708 Mannedorf), or the Synergy™ 2 model microplate reader equipped with reagent dispensers (BioTek, Winooski, Vt. 05404).

In one embodiment, the method of the present invention is based on a process in which a gas (e.g. carbon dioxide) or a mixed gas (e.g., containing nitrogen and carbon dioxide) is supplied to the headspace of one or more reaction vessels containing the assay liquid. Once the CO₂ is passed from the gas into the liquid, equilibrium between bicarbonate, carbonic acid, dissolved CO₂, and carbonate will be established in the liquid phase. Preferably, the gas-liquid interface between the headspace and the assay liquid in the reaction vessel has a high surface area to facilitate a large area of gas-liquid contact allowing as much gaseous CO₂ to interact with the assay liquid as possible. A large surface area can, e.g., be obtained by using a low liquid volume in a wide reaction vessel, and/or using a reaction vessel fabricated from material that allows the assay liquid to spread on the reactor vessel surface, preferably uniformly.

In one embodiment of using the method for analysis of the desorption reaction, the assay liquid is initially enriched in bicarbonate or a combination of bicarbonate and carbonic acid. The reaction of converting bicarbonate in the liquid into CO₂ preferably takes place when the initial pH is sufficiently low and/or a driving force, such as heat, or low partial pressure of CO₂ in the headspace is present. Conversion of carbonic acid to bicarbonate, bicarbonate to carbonate, and/or release of CO₂ from the assay liquid will result in an increased pH of the assay liquid, which can be detected by monitoring the absorbance or color change of the dissolved pH indicator. This process of converting bicarbonate in the liquid into CO₂ involves dehydration of the bicarbonate and is, therefore, termed the dehydration reaction. Similarly, the absorption reaction may be termed the CO₂ hydration reaction in the event where CO₂ is converted into bicarbonate and/or carbonic acid.

The CO₂ may pass in and out of the liquid phase by diffusion (heat-, pH-, or pressure-aided) and/or the transfer may be aided by an enzyme or a chemical or physical compound that has affinity toward CO₂. One exemplary enzyme is carbonic anhydrase. Since carbonic anhydrase reacts specifically with dissolved CO₂, it favors the movement of gaseous CO₂ into the fluid in the absorption reaction by accelerating the reaction of the dissolved CO₂ and water to form an equilibrium mixture of carbonic acid, bicarbonate, and carbonate, depending on the pH, thereby removing CO₂ rapidly and allowing the dissolution of more CO₂ from the headspace gas into the aqueous assay liquid to a greater extent than would occur only by diffusion. Likewise carbonic anhydrase will catalyze the reverse reaction, termed the desorption/dehydration reaction, converting bicarbonate into CO₂ which will result in an increase pH and potentially a release of CO₂ from the assay liquid under the conditions of an appropriate driving force, such as heat or low CO₂ partial pressure in the headspace.

The biocatalyst carbonic anhydrase or a chemical catalyst used to facilitate the CO₂ absorption into the assay liquid may either be dissolved in solution in the assay liquid and/or may be floating at the surface of the liquid, and/or may be suspended as particles or aggregates in the liquid, and/or may be immobilized on carrier materials, such as solid or porous beads, and/or may be affixed on or entrapped in any non-soluble or semi-soluble (e.g. a gel) material that is prepared in sufficiently small size to be dosed into the reaction vessel, and/or may be immobilized on the surface of the reactor vessel. Immobilization can, for example, be achieved by cross-linking and/or by affixing a gel or polymer matrix containing the carbonic anhydrase or chemical onto the reactor surface or other non-soluble or semi-soluble surface exposed to the assay liquid in the reaction vessel. In one embodiment the biocatalyst (e.g., carbonic anhydrase) is present in the reaction vessel together with a compound (e.g. bicine) that provides buffer capacity for CO₂ hydration or dehydration to occur.

The equipment design of the present invention provides increased: (i) throughput and reagent savings, resulting from the use of a 96-well microtiter plate format and the high speed and ease of data processing as a result of using the automated data processing template, (ii) reliability of results, resulting from the ability to run many replicates and having controlled and reproducible timing of assay steps (such as liquid dispense and read times), (iii) ability to dose CO₂ and run the assay at application relevant conditions due to fast optics and instrument-based data handing which allows measurement of the CO₂ absorption reactions in temperature ranges at or near the application temperature (for example in the range 20-40° C.) where the reaction occurs much more rapidly than at the non-application relevant cold temperatures (such as around 4° C.) used in conventional published methods.

Although the temperature conditions may be limited by physical constraints of currently available equipment, in one embodiment of the present invention the assay chamber of the equipment is maintained at a temperature of between 0-100° C., between 0-80° C., between 0-70° C., between 0-50° C., or between 20-40° C. The temperature at which the assay is operated will be dependent on the temperature constraints of the equipment. Within the constraints of the equipment, the selected assay temperature may be a temperature similar to the temperature the catalyst will experience in the targeted application. The temperature can be regulated by cooling or heating the assay liquids and, optionally, the gas in the headspace. Optionally, heating and cooling may be applied only within the reaction vessel, such as achieved with a localized heating element, or to the entire equipment assembly, such as achieved by locating the equipment in a temperature-controlled environment, such as an enclosed environmental chamber. Humidity in the headspace can be controlled by conducting the assay in an enclosed environmental chamber. Humidity control may be desirable when conducting the assay at elevated temperature, to control the evaporation of water from the assay liquid. In a reaction assay the temperature may be adapted to the optimum temperature of the catalyst, such as an enzyme catalyst, present in the reaction vessel. Normally mammalian, plant and prokaryotic carbonic anhydrases function at 37° C. or lower temperatures. However, WO 2008/095057, US 2006/0257990, US 2008/0003662, WO 2010/151787, and WO 2012/025577 describe heat-stable carbonic anhydrases (the content of these applications is hereby incorporated by reference). In one embodiment of the present invention, a heat-stable carbonic anhydrase is applied in a reaction assay of the present invention.

In the assay methods of the present invention, one or more carbonic anhydrase (EC 4.2.1.1) can be used as a CO₂ absorption catalyst. For example, one or more of the previously described carbonic anhydrases or a carbonic anhydrase described in the section “Enzymes for the method” is used in the method. In a further embodiment of the present invention, the assay method comprises two or more different carbonic anhydrase enzymes. Although any relevant amount of carbonic anhydrase can be applied in the assay methods, in some embodiments the amount of carbonic anhydrase is below 2 g enzyme protein/L assay liquid, e.g., below 1.5 g/L, below 1 g/L, below 0.6 g/L, below 0.3 g/L, below 0.1 g/L, below 0.05 g/L, below 0.01 g/L, below 0.005 g/L, below 0.001 g/L or below 0.0005 g/L.

When the test sample to be analyzed is very dilute, it may be necessary to adjust the sample pH before diluting due to the dilution factor used in the method for dilute samples will be correspondingly smaller, resulting in less buffer capacity provided by the assay buffer diluent. Alternatively, in cases where the buffer capacity of the test sample itself overwhelms the buffer capacity of the assay buffer, dialysis or similar methods known in the art may be used to correct the test sample buffer capacity and/or composition to a level that can be tolerated by the assay buffer. In the event of such adjustments, any relevant additional dilution factors should be accounted for when reporting the assay result.

### Absorption Liquids

The methods of the present invention may also comprise an assay liquid with a chemical or physical solvent that has affinity toward CO₂ to facilitate CO₂ absorption and/or desorption. Such chemicals can, e.g., constitute conventional CO₂ absorption compounds such as chemical absorption via amine-based solvents or aqueous ammonia or blends of such chemicals. Physical CO₂ solvents can, e.g., be Selexol™ (Union Carbide) or water or glycerol or polyethylene glycol ethers, or polyethylene glycol dimethyl ether. Carbonic anhydrase may be combined with these conventional CO₂ absorption solvents. PCT/US2008/052567 shows that by adding carbonic anhydrase to a MEA solution the efficiency of the CO₂ absorption is significantly increased and the amount of carbonic anhydrase can be reduced at least two times. In a further embodiment of the present invention the assay liquid comprises a carbonic anhydrase in combination with one or more carbon dioxide absorbing compound(s) such as amine-based compounds such as aqueous alkanolamines including monoethanolamine (MEA), diethanolamine (DEA), methyldiethanolamine (MDEA), 2-amino-2-methyl-1-propanol (AMP), 2-amino-2-hydroxymethyl-1,3-propanediol (AHPD), Tris or other primary, secondary, tertiary or hindered amine-based solvents such as piperazine and piperidine and derivatives of these, or polyethylene glycol ethers, or aqueous salts of amino acids such as glycine or derivatives of these such as taurine or other liquid absorbers such as aqueous NaOH, KOH, LiOH, phosphate, carbonate or bicarbonate solutions at different ionic strengths or aqueous electrolyte solutions, or a blend of them or analogs or blends thereof. An example of a carbonate-based CO₂ absorption liquid is an aqueous solution comprising carbonic anhydrase and 20 wt % potassium carbonate (Lu, Y. et al. 2011. Energy Procedia 4: 1286). See also WO2011/014955, the content of which is hereby incorporated by reference.

Aqueous electrolyte solutions may comprise salts, such as salts comprised of halides and alkali metals, for example fluorides, chlorides, bromides, or iodides of lithium, sodium, or potassium, or halide salts of metals, such as LiCI, KCl, NaCl, ZnCl₂, and ZnSO₄. In another embodiment, aqueous electrolyte solutions may comprise carbonate, bicarbonate, sulfate, phosphate, or nitrate salts of alkali metals, such as potassium sulfate or sodium sulfate.

Suitable concentrations for the carbon dioxide absorbing compound(s) range from 0.1 M to greater than the solubility limit of the compounds for systems operating solid sorbents or solid/liquid slurries. An advantage to operating water deficient systems is a decreased thermal energy requirement for CO₂ absorbing compound regeneration. For aqueous solvent systems, suitable concentrations range from 0.1 M to the solubility limit of the CO₂ absorbing compound(s), and, e.g., in a range from 1 to 5 M, and e.g., a CO₂-lean liquid pH for absorption of pH>8, e.g., pH>9 or pH>10.

An aspect of the present invention is to include one or more salts in the CO₂ absorption enhancement measurement of the present invention. In one embodiment, the method of the present invention can be used to measure the CO₂ absorption enhancement provided by a salt compound, and/or compare among the performance of different salt compounds as CO₂ absorption enhancement compounds. In another embodiment, the method of the present invention can be used to measure the CO₂ absorption enhancement provided by carbonic anhydrase in the presence of a salt compound, and/or compare among the CO₂ absorption enhancement provided by carbonic anhydrase in the presence of different salt compounds, or in the presence of mixtures of salt compounds. In another embodiment the method of the present invention can be used to measure the CO₂ desorption enhancement provided by a salt in the presence or absence of carbonic anhydrase, and/or compare among the performance of different salts together with carbonic anhydrase as CO₂ desorption enhancement compounds. The concentrations of carbonic anhydrase or the different salt compounds can be manipulated to optimize the CO₂ absorption or desorption enhancement. In another embodiment of the present invention, one or more salt compounds are included together with one or more carbonic anhydrases in a CO₂ absorption solution. In one embodiment, a salt mixture comprises bicine and a salt described herein (e.g., potassium carbonate, LiCI, KCl, NaCl, ZnCl₂, and/or ZnSO₄).

An aspect of the present invention is to include one or more surfactants in the CO₂ absorption enhancement measurement of the present invention. The surfactant may be nonionic including semi-polar and/or anionic and/or cationic and/or zwitterionic. Nonionic surfactants include but are not limited to alkyl polyethylene oxide, alkylphenol polyethylene oxide, copolymers of polyethylene oxide and polypropylene oxide (commercially called Poloxamers or Poloxamines), alkyl polyglucosides such as octylglucoside, fatty alcohols such as cetyl alcohol and oleyl alcohol, polysorbates, such as Tween20 and Tween80, dodecyl dimethylamine oxide, alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”). Anionic surfactants include but are not limited to perflurooctanoic acid, sodium dodecyl sulfate, ammonium lauryl sulfate, and other alkyl sulfate salts, alkyl benzene sulfonate, linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl-or alkenylsuccinic acid and soap. Cationic surfactants include, but are not limited to cetyl trimethylammonium bromide (CTAB) such as hexadecyl trimethyl ammonium bromide and other alkyltrimethylammonium salts, cetylpyridinium chloride (CPC), polyethoxylated tallow amine (POEA), benzalkonium chloride (BAC) and benzethonium (BZT). Zwitterionic surfactants include, but are not limited to dodecyl betaine, cocamidopropyl betaine, and coco ampho glycinate. The surfactant may also contain PEG/VA polymers, ethoxylated (EO) or propoxylated (PO) polymers such as EO/PO polyethyleneimine, EO/PO polyamidoam-ine or EO/PO polycarboxylate (described in EP 1876227). Low-foaming nonionic surfactants are preferred for such application. Alkyl-capped non-ionic surfactants Cₙ(EO)ₘ is in this category. Also preferred are EO/PO block copolymers and certain silicone based surfactants or lubricants. Examples of commercially available surfactants are Ethox L-61, Ethox L62 and Ethox L64 (Ethox, Greenville, S.C. USA). In one embodiment, surfactant is present in the assay liquid. In another embodiment, surfactant is present in the test sample. In one embodiment, the method of the present invention can be used to measure the CO₂ absorption enhancement provided by a surfactant, and/or compare among the performance of different surfactants as CO₂ absorption enhancement compounds. In another embodiment the method of the present invention can be used to measure the CO₂ desorption enhancement provided by a surfactant, and/or compare among the performance of different surfactants as CO₂ desorption enhancement compounds. In another embodiment of the present invention, one or more surfactants are included together with one or more carbonic anhydrases in a CO₂ absorption solution. In another embodiment of the present invention, one or more surfactants and one or more salts are included together with one or more carbonic anhydrases in a CO₂ absorption solution.

The CO₂ desorption rate from a particular volume of liquid can be increased by increasing the area of the gas-liquid interface. This can either be done by using a reaction vessel with a large gas-liquid surface area to liquid reaction volume ratio.

In certain embodiments of the present invention, one or more antifoam/defoaming agents is added for the CO₂ absorption enhancement measurement of the present invention. Antifoam/defoaming agents include but are not limited to silicone oils or silicone oil based emulsions, mineral oils or mineral oil based emulsions, vegetable oils or vegetable oil based emulsions, polyalkylene glycols or polyalkylene glycol based emulsions, and fatty acids or fatty acid based emulsions.

### Uses

The methods of the present invention are useful for measuring the CO₂ absorption enhancing performance of compounds, especially catalysts. In one embodiment, methods of the present invention are useful for measurement of carbonic anhydrase activity. Measuring catalyst activity is useful for selecting the best-performing catalyst from a group. The methods are also useful for monitoring catalyst performance as a result of exposure to different conditions. The activity in this case is often expressed as percent residual activity, where the ratio of activity measured after the exposure versus the activity measured before the exposure is expressed as a percent. The methods are furthermore useful for identifying whether a sample, such as an enzyme sample, is able to catalyze CO₂ absorption. Because the methods can be performed across a range of temperatures, such as from a cold room condition of around 4° C. to a heated condition such as around 40-60° C., which may be a limitation of the equipment, the methods can be used to directly compare catalyst performance at different temperatures. In this case, the different solubility of CO₂ in water at the different assay temperatures may need to be taken into account. The linearity test (as described herein) will identify whether non-substrate saturating conditions have occurred, potentially as a result of the temperature, such as an elevated temperature, chosen for the test. Use of the methods at higher temperatures, for example above approximately 40-60° C., could be possible if the equipment is manufactured to perform at higher temperatures, and the lower solubility of CO₂ at elevated temperatures could be overcome by installing the equipment in a chamber, such as an environmental chamber, where the partial gas pressure of CO₂ inside the chamber is increased relative to the ambient CO₂ partial pressure outside the chamber. The methods could alternatively be carried out at the lower temperatures mentioned (e.g. in the range 4-50° C.) in an environmental chamber with increased CO₂ partial pressure in order to increase the saturation of CO₂ in the assay substrate. Furthermore, by comparison of blanks versus modified blanks (as defined herein), the methods can be used to determine whether components that interfere with the CO₂ absorption enhancing effect of the component of interest, such as carbonic anhydrase, are present. When performed in a desorption configuration, the methods can be used to measure CO₂ desorption enhancement of compounds, such as catalysts, and especially enzymes such as carbonic anhydrase.

### Enzymes for the Method

In some embodiments, the enzyme for the methods of the present invention is carbonic anhydrase.

Carbonic anhydrases (CA, EC 4.2.1.1, also termed carbonate dehydratases) catalyze the inter-conversion between carbon dioxide and bicarbonate [CO₂+H₂O⇄HCO₃⁻+H⁺]. The enzyme was discovered in bovine blood in 1933 (Meldrum and Roughton, 1933, J. Physiol. 80: 113-142) and has since been found widely distributed in nature in all domains of life from mammals, plant, fungi, bacteria and archaea. Carbonic anhydrase enzymes are categorized in three distinct classes called the alpha-, beta- and gamma-class, and potentially a fourth class, the delta-class. There are several sources of carbonic anhydrase, e.g., the mammalian alpha carbonic anhydrases CA-I or CA-II isolated from human or bovine erythrocytes which can be purchased commercially. US 2006/0257990 describes a variant of human carbonic anhydrase with increased thermostability. The gamma carbonic anhydrase, CAM, from Methanosarcina thermophila strain TM-1 (DSM 1825) is also well described (Alber and Ferry, 1994, Proc. Natl. Acad. Sci. USA 91: 6909-6913). WO 2008/095057 and U.S. Application No. 61/220,636 describe heat-stable alpha-carbonic anhydrase from bacteria. Any of these enzymes or blends of these enzymes may be used in the methods and compositions of the present invention. Exemplary heat-stable carbonic anhydrases for the methods and compositions of the present invention are SEQ ID NO: 2, 4, 6, 8, 10, 12, 14 or 16 from WO 2008/095057 (hereby incorporated by reference) or SEQ ID NO: 2 of U.S. application No. 61/220,636 (hereby incorporated by reference). In one embodiment, methods of the present invention may be used to identify particular carbonic anhydrases that preferentially catalyze the CO₂ hydration reaction. In another embodiment, methods of the present invention may be used to identify particular carbonic anhydrases that preferentially catalyze the dehydration of carbonic acid or bicarbonate (also referred to herein as the CO₂ dehydration reaction).

For certain applications, immobilization of the carbonic anhydrase may be preferred. An immobilized enzyme comprises two essential functions, namely the non-catalytic functions that are designed to aid separation (e.g., isolation of catalysts from the application environment, reuse of the catalysts and control of the process) and the catalytic functions that are designed to convert the target compounds (or substrates) to products within the time and space desired (Cao, Carrier-bound Immobilized Enzymes: Principles, Applications and Design, Wiley-VCH Verlag GmbH & Co. KGaA, Weinheim, Germany, 2005). When an enzyme is immobilized it is made insoluble to the target compounds (e.g., substrates) it aids converting and to the solvents used. An immobilized enzyme product can be separated from the application environment in order to facilitate its reuse, or to reduce the amount of enzyme needed in the application environment, or to use the enzyme in a process where substrate is continuously delivered and product is continuously removed from proximity to the enzyme, which, e.g., reduces the amount of enzyme needed per amount substrate converted. Furthermore, enzymes may be stabilized by immobilization which can allow the enzyme to operate longer in the application. A process involving immobilized enzymes is often continuous, which facilitates easy process control. The immobilized enzyme can be restrained by physical means, such as by entrapment of the enzyme in a space in such a way that the enzyme cannot move away from that space. For example, this can be done by entrapping the enzyme in a polymeric cage, wherein the physical dimensions of the enzyme are too large for it to pass between adjacent polymer molecules forming the cage. Entrapment can also be done by confining the enzyme behind membranes that allow smaller molecules to pass freely while retaining larger molecules, e.g., using semi permeable membranes or by inclusion in ultrafiltration systems using, e.g., hollow fiber modules, semi permeable membrane stacks, etc. Immobilization on porous carriers is also commonly used. This includes binding of the enzyme to the carrier, e.g., by adsorption, complex/ionic/covalent binding, or just simple absorption of soluble enzyme on the carrier and subsequent removal of solvent. Cross-linking of the enzyme can also be used as a means of immobilization. Immobilization of enzyme by inclusion into a carrier is also industrially applied. (Buchholz et al., Biocatalysts and Enzyme Technology, Wiley-VCH Verlag GmbH & Co. KGaA, Weinheim, Germany, 2005). Specific methods of immobilizing enzymes such as carbonic anhydrase include, but are not limited to, spraying of the enzyme together with a liquid medium comprising a polyfunctional amine and a liquid medium comprising a cross-linking agent onto a particulate porous carrier as described in WO 2007/036235 (hereby incorporated by reference), linking of carbonic anhydrase with a cross-linking agent (e.g., glutaraldehyde) to an ovalbumin layer which in turn adhere to an adhesive layer on a polymeric support as described in WO 2005/114417 (hereby incorporated by reference), or coupling of carbonic anhydrase to a silica carrier as described in U.S. Pat. No. 5,776,741 or to a silane, or a CNBr activated carrier surface such as glass, or co-polymerization of carbonic anhydrase with methacrylate on polymer beads as described in Bhattacharya et al., 2003, Biotechnol. Appl. Biochem. 38: 111-117 (hereby incorporated by reference). In an embodiment of the present invention carbonic anhydrase is immobilized on a matrix. The matrix may, e.g., be selected from the group beads, fabrics, fibers, hollow fibers, membranes, particulates, porous surfaces, rods, structured packing, and tubes. Specific examples of suitable matrices include alumina, bentonite, biopolymers, calcium carbonate, calcium phosphate gel, carbon, cellulose, ceramic supports, clay, collagen, glass, hydroxyapatite, ion-exchange resins, kaolin, nylon, phenolic polymers, polyaminostyrene, polyacrylamide, polypropylene, polymerhydrogels, sephadex, sepharose, silica gel, precipitated silica, and TEFLON-brand PTFE. In an embodiment of the present invention carbonic anhydrase is immobilized on a nylon matrix according to the techniques described in Methods in Enzymology, Volume XLIV (section in the chapter: Immobilized Enzymes, pages 118-134, edited by Klaus Mosbach, Academic Press, New York, 1976), hereby incorporated by reference.

The carbonic anhydrase to be evaluated by the method may be stabilized in accordance with methods known in the art, e.g., by adding an antioxidant or reducing agent to limit oxidation of the carbonic anhydrase or it may be stabilized by adding polymers such as PVP, PVA, PEG, sugars, oligomers, polysaccharides or other suitable polymers known to be beneficial to the stability of polypeptides in solid or liquid compositions. A preservative, such as penicillin, virginiamycin, or Proxel™ (Arch Chemicals, Inc.), can be added to extend shelf life or performance in application by preventing microbial growth.

The assays of the present invention can be used to identify various properties of the catalyst, e.g., carbonic anhydrases. It is known in the art that most carbonic anhydrases require zinc at the active site for catalytic activity. Removal of zinc has been shown to result in loss of activity and the removed zinc can be reintroduced to regain activity (Lindskog, S. and B. G. Malmstrom. 1960. Biochemical and Biophysical Research Communications. 2: 213). In a particular embodiment, as exemplified in Example 6, the assays of the present invention were used to determine that zinc (e.g., ZnCl₂ or ZnSO₄) may be added to improve the enzymatic activity of a carbonic anhydrase. In a particular embodiment, the present invention provides methods for improving the activity of a carbonic anhydrase comprising adding zinc to a composition comprising one or more carbonic anhydrases. Such methods may be applied in the context of a method for carbon dioxide (CO₂) absorption. The invention also relates to compositions comprising one or more carbonic anhydrases and zinc ions, wherein the zinc ions were added to the composition independent of the natural zinc content of the one or more carbonic anhydrases, or wherein the zinc ions are added to the composition independently of the zinc content of the composition comprising one or more carbonic anhydrases produced by fermentation methods. For example, in cases where carbonic anhydrase produced by fermentation methods results in inactive enzyme molecules, potentially due to insufficient zinc added during fermentation or due to inadequate uptake of zinc by the production organism, zinc can be added to activate the inactive molecules after the completion of fermentation, either to the fermentation broth, or in a separate step during recovery of the enzyme after fermentation, or at a later point independent of enzyme production, such as addition to an enzyme sample that has been stored for a period of time. The invention also relates to addition of zinc ions to a CO₂ absorbing solution, whereby the addition of zinc improves and/or extends the performance of carbonic anhydrase in the CO₂ absorbing solution. The zinc ions are added in an amount effective to increase the catalytic activity of a carbonic anhydrase and/or extend the longevity of the catalytic activity.

In another particular embodiment, as exemplified in Example 2, the assay methods of the present invention were used to demonstrate that addition of salt, e.g. NaCl, can improve the activity of a carbonic anhydrase test sample. Halophilic (enzymes that require salt compounds for optimal activity) as well as halotolerant (enzymes that demonstrate optimal activity over a range of different salt concentrations) carbonic anhydrases are known in the art (Premkumar, L. et al., 2005, PNAS, 102(21): 7493-7498). The assay methods of the present invention can be used to measure the salt-dependency of different carbonic anhydrases.

### Compositions

The present invention also relates to improved carbonic anhydrase compositions comprising an aqueous composition comprising one or more carbonic anhydrases and a buffer compound comprising an N,N-disubstituted derivative of an amino acid which comprises a tertiary amine functional group (and which, in some embodiments, does not comprise a primary or a secondary amine functional group), wherein the buffer is added in an amount effective to permit measuring the activity of the one or more carbonic anhydrases. In some embodiments, the N,N-disubstituted derivative comprises at least hydroxy-substituted alkanyl moiety, e.g., a hydroxymethyl, hydroxyethyl, hydroxylpropyl, and/or hydroxylisopropyl moiety.

The present invention also relates to improved carbonic anhydrase compositions comprising an aqueous composition comprising one or more carbonic anhydrases and bicine, wherein the bicine is added in an amount effective to permit measuring the activity of the one or more carbonic anhydrases.

The present invention also relates to improved carbonic anhydrase compositions comprising an aqueous composition comprising one or more carbonic anhydrases and a buffer comprising an N,N-disubstituted derivative of an amino acid which comprises a tertiary amine functional group, e.g., bicine, wherein the buffer is added in an amount effective to provide CO₂ absorption, such as is required for removal of CO₂ from a mixed gas, where the rate of CO₂ absorption into the aqueous buffer containing composition or the rate of CO₂ desorption out of the aqueous buffer containing composition is catalyzed by the one or more carbonic anhydrases. Amino acids, and derivatives of amino acids as described in the present invention, have both a carboxylic acid functional group and an amine group in the molecular structure, which can attain a zwitterionic state in aqueous solution, wherein the amine group is protonated and the carboxylic acid group is deprotonated. This zwitterionic feature provides buffer capacity as well as CO₂ absorption. Increasing the concentration in aqueous solution of buffers described herein increases the buffer capacity as well as the CO₂ absorption of the aqueous solution.

The compositions may include, for example, any of the embodiments described supra, e.g., any of the embodiments envisioned above for Absorption liquids.

### Methods of Carbonic Anhydrase Activity Analysis

### Measurement of Carbonic Anhydrase Activity Using a Manual Bromothymol Blue-Based Method

A method for the measurement of carbonic anhydrase activity has been described by Wilbur, 1948, J. Biol. Chem. 176: 147-154. The method is based on the pH change of the assay mixture due to the formation of bicarbonate from carbon dioxide (described above).

Described here is a version of the Wilbur method derived from the procedure of Chirica et al., 2001, Biochim. Biophys. Acta 1544(1-2): 55-63. An aqueous solution containing approximately 60 to 70 mM CO₂ was prepared by bubbling CO₂ through the tip of a syringe into 100 mL distilled water for approximately 45 min to 1 h prior to the assay. The aqueous CO₂ substrate was chilled in an ice-water bath. To test for the presence of carbonic anhydrase, 2 mL of 25 mM Tris, pH 8.3 (containing sufficient bromothymol blue to give a distinct and visible blue color) were added to two 13×100 mm test tubes chilled in an ice bath. To one tube, 10 to 50 microliters of the enzyme containing solution (e.g., culture broth or purified enzyme) was added, and an equivalent amount of buffer was added to the second tube to serve as a blank. Using a 2 mL syringe and a long cannula, 2 mL of aqueous CO₂ substrate was added quickly and smoothly to the bottom of each tube. Simultaneously with the addition of the aqueous CO₂ substrate, a stopwatch was started. The time required for the solution to change from blue to yellow by visual observation was recorded (transition point of bromothymol blue is pH 6-7.6). The production of hydrogen ions during the CO₂ hydration reaction lowers the pH of the solution until the color transition point of the bromothymol blue is reached. The time required for the color change is inversely related to the quantity of carbonic anhydrase present in the sample. The tubes remain immersed in the ice bath for the duration of the assay for results to be reproducible. Typically, the uncatalyzed reaction (the control) takes approximately 2 min for the color change to occur, whereas the enzyme catalyzed reaction is complete in 5 to 15 sec, depending upon the amount of enzyme added. Detecting the color change is somewhat subjective but the error for triple measurements was in the range of 0 to 1 sec difference for the catalyzed reaction. One unit is defined after Wilbur [1 U=(1/t_{c})−(1/tᵤ)×1000] where U is units and t_{c} and tᵤ represent the time in seconds for the catalyzed and uncatalyzed reaction, respectively (Wilbur, 1948, J. Biol. Chem. 176: 147-154). These units are also termed Wilbur-Anderson units (WAU). Drawbacks of this method are the reliance on visual observation to determine the color change end-point (which may differ from one observer to the next), the need to conduct the reaction at cold temperature (to sufficiently slow down the reaction that a human observer can monitor the time with a stopwatch), the long time required to collect replicate measurements (especially for the blank reactions), manual recording of the data, and the relatively large volumes of reagents needed.

Measurement of Carbonic Anhydrase Activity Using a p-Nitrophenyl Acetate Based Method

This method utilizes para-nitrophenol acetate (pNP-acetate) as a colorimetric substrate for carbonic anhydrase activity. The analysis method is therefore an indirect measure of carbonic anhydrase CO₂ hydration activity because the method assumes that the hydrolysis of pNP-acetate in the enzyme active site, which is a measure of esterase activity, correlates to the enzyme's CO₂ hydration activity. Twenty microliters of purified carbonic anhydrase (CA) enzyme sample (diluted in 0.01% Triton X-100) was placed in the bottom of a micro-titer plate (MTP) well. The assay was started at room temperature by adding 200 microliters para-nitrophenol-acetate (pNP-acetate, Sigma, N-8130) substrate solution in the MTP well. The substrate solution was prepared immediately before the assay by mixing 100 microliters pNP-acetate stock solution (50 mg/ml pNP-acetate in DMSO. Stored frozen) with 4500 microliters assay buffer (0.1 M Tris/HCl, pH 8). The increase in OD₄₀₅ was monitored over a fixed interval of time. In the assay, a buffer blank (20 microliters assay buffer instead of CA sample) was included. The difference in OD₄₀₅ increase between the sample and the buffer blank was a measure of the carbonic anhydrase activity (CA activity=ΔOD₄₀₅(sample)−ΔOD₄₀₅(buffer)). Key drawbacks of this method are the assumption that pNP-acetate activity (measuring ester hydrolysis activity) of a particular carbonic anhydrase sample correlates to CO₂ hydration activity, and that the carbonic anhydrase samples does not contain side-activities, such as lipase activity, that could influence the level of pNP-acetate hydrolysis. According to published reports, there are cases where carbonic anhydrase activity measured according to the pNP-acetate method does not correspond to carbonic anhydrase activity measured according to a CO₂ hydration method (Premkumar, L. et al., 2005. PNAS, 102(21): 7493-7498).

### EXAMPLES

### Example 1

### Measurement of CO₂ Absorption Enhancement by Carbonic Anhydrase Using 25 mM Bicine and Cresol Red

A solution containing 25 mM bicine and 123 micromolar (0.05 g/L) cresol-red (pH 8.3) is prepared as the assay reagent. The assay substrate is prepared at room temperature by bubbling CO₂ gas into either a 125 mL Erlenmeyer flask containing 100 mL deionized H₂O (dH₂O) or a 50 ml plastic conical tube containing 30 mL dH₂O. Bubbling proceeds for at least 10 minutes to yield a saturated CO_{2(aq)} solution ([CO₂]˜30 mM (Kernohan, J. C. 1965. Biochim. Biophys. Acta 96: 304). Carbonic anhydrase test samples are diluted for the assay using the assay reagent as diluent. A set of assay samples, made by 5- or 6-×0.7-fold serial dilutions of the enzyme test sample, are loaded onto a 96-well microtiter plate. Eight replicate wells each containing 100 microliters of each dilution are plated. Corresponding blank wells are loaded on the same plate in alternating columns with the assay sample wells. Blank wells contain either assay reagent alone or a 5- or 6-×0.7-fold serial dilution set of a modified enzyme test sample, where the modification involves removing or inactivating the active enzyme of the test sample.

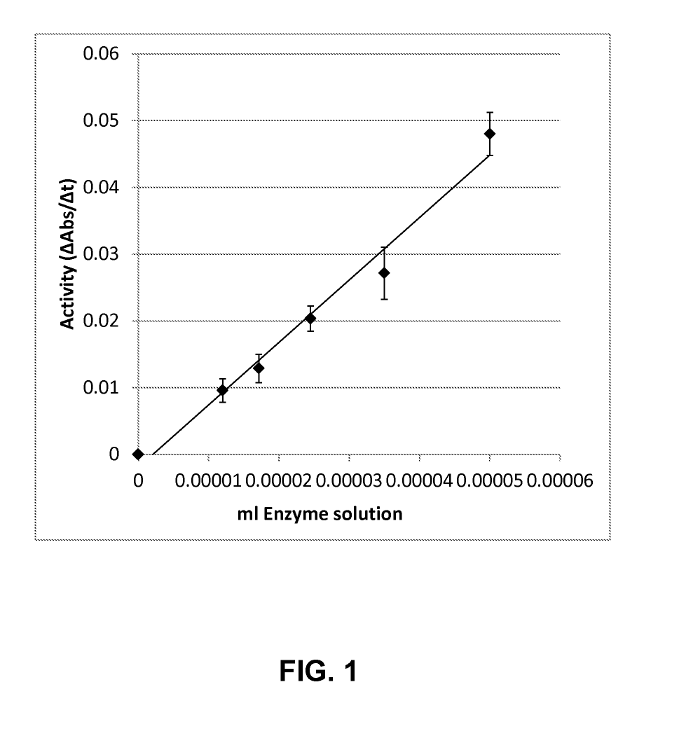

The plate is inserted into a temperature controlled plate reader equipped with a liquid dispenser unit with the inject head immersed in the aqueous CO₂ substrate solution. Initial absorbance of a single well is collected, followed by injection of 100 microliters of aqueous CO₂ substrate into a single well and kinetic absorbance reads are collected at fixed time intervals (e.g., every 0.5 seconds) for that well such that the rate of absorbance change can be measured and used to indicate the rate of CO₂ hydration. After approximately 5-10 seconds the reaction is no longer monitored and the process is repeated for all subsequent wells. Data automatically collected on the instrument is saved or exported in Microsoft Excel file format and copy/pasted into the Microsoft Excel automated data processing template. The data collected for each well is automatically checked for a linear slope (change in absorbance with change in time) within a selected “assay window” indicating the enzyme in that well was saturated with substrate during the assay window. The assay window is a sub-set of data points collected between selected time points for each well. The assay window for all wells in this example are the same. The slope of the data collected from an individual well assay window is called the “assay slope.” Wells passing the “linearity test” are averaged for a particular dilution to determine the “average assay slope” for that particular dilution. Next, a T-test between blank and assay sample replicates determines the probability that the differences between the two data sets could arise by chance, improving data quality by ensuring that data from an excessively diluted assay sample are not included in calculations of enzyme activity. If at least 3 dilution sets pass the T-test (T-test value <0.05), and at least 4 out of 8 replicate wells for those dilutions pass the “linearity test,” the average assay slope (change in absorbance per unit time) for each accepted dilution is plotted versus the amount of enzyme present in that dilution. The slope of the “average assay slope” versus enzyme amount (see FIG. 1) line corresponds to the activity of the carbonic anhydrase sample and is reported in units per unit amount of enzyme or enzyme containing solution. For example, the amount of enzyme can be reported as mL of enzyme sample, or milligrams of enzyme protein (such as when the concentration of enzyme protein in a liquid test sample is known).

Benefits of this method compared to the manual reference case includes: (i) confidence that enzyme activity metrics considered for activity determination are taken under substrate saturating conditions, (ii) measuring rates instead of time required to reach an endpoint, (iii) assaying activity at a more application relevant temperature (vs. ice bath), (iv) increased throughput, and (v) more replicates for each dilution tested.

Table 1 shows a comparison of a sub-set (the first two wells only, one for the assay sample and one for the corresponding blank) of assay results for each of three different microtiter plates. The three microtiter plates each corresponded to a different test sample of carbonic anhydrase enzyme that had been diluted to different levels. The sub-set of data was selected to illustrate the linearity test and T-test that are used for acceptance or rejection of data. For ease of illustration, the data in the sub-set does not represent the full data set collected for the three microtiter plates. The Enz-A sample dilution was determined to be too concentrated due to the Linear Ratio (measured by comparing the assay window time points between 5.1 to 6.7 seconds, inclusive) is greater than 1.18 (the upper allowable limit of the Linear Ratio range established for the method of the present example). In this case the T-test is not relevant because the data failed the linearity test. The Enz-B sample dilution was determined to be too dilute due to the high value of the T-test (0.54, based on utilization of the full data set, not shown) which indicates there is no statistical basis for asserting that the data for Blank-B is different than the data for Enz-B. Therefore, although the Linear Ratio was within acceptable limits (near 1), the data was rejected based on failing the T-test. A visual comparison of the presented data supports the statistical conclusion. For example, a comparison of the data at the time point 5.9 seconds shows a large difference between the absorbance values of Blank-A and Enz-A, but only a small difference between the absorbance values of Blank-B and Enz-B. The Enz-C dilution was determined to be acceptable for the assay because the Linear Ratio fell with the allowable range (0.85-1.18) established for the method of the present example, and because the full data set (not shown) passed the T-test (with a value of 5.6×10⁻⁷). This example further illustrates the importance of including appropriate blanks on each assay microtiter plate. Comparison of blank absorbance values at time point 0 seconds shows that initial values for blank absorbance can change from one plate to the next, such as may be caused by fresh preparation of assay reagent.

FIG. 1 presents enzyme activity (change in absorbance with change in time) as a function of enzyme solution volume, based on calculations utilizing the full data set (not shown) for Enz-C. Only dilutions that passed both the “linearity test” and the T-test are included in the graph. The slope of this line is reported as the enzyme activity. Error bars represent standard error of the mean (SEM).

TABLE 1
Absorbance versus time for three different
of sample dilutions of carbonic anhydrase

| Time (s) | Blank-A | Enz-A | Blank-B | Enz-B | Blank-C | Enz-C |
|---|---|---|---|---|---|---|
| 0 | 0.95 | 0.85 | 0.78 | 0.74 | 0.88 | 0.87 |
| 4.3 | 0.83 | 0.63 | 0.62 | 0.64 | 0.69 | 0.54 |
| 5.1 | 0.78 | 0.47 | 0.59 | 0.60 | 0.62 | 0.44 |
| 5.9 | 0.72 | 0.35 | 0.54 | 0.56 | 0.57 | 0.35 |
| 6.7 | 0.66 | 0.26 | 0.49 | 0.52 | 0.53 | 0.27 |
| 7.4 | 0.60 | 0.20 | 0.44 | 0.49 | 0.48 | 0.20 |
| 8.2 | 0.56 | 0.15 | 0.40 | 0.44 | 0.44 | 0.15 |
| 9 | 0.51 | 0.11 | 0.36 | 0.40 | 0.41 | 0.11 |
| Linear Ratio | 1.02 | 1.28 | 0.98 | 1.09 | 1.13 | 1.15 |

### Example 2

### Measurement of CO₂ Absorption Enhancement by Carbonic Anhydrase Using 50 mM Bicine and Cresol Red

The measurement of CO₂ absorption enhancement by carbonic anhydrase was performed as described in Example 1 except the assay reagent contains 50 mM bicine at pH 8.65+/−0.05. This embodiment of the assay reagent delays the assay response relative to Example 1. Such a delay expands the “assay window” (from 5.1 to 6.7 seconds, inclusive, in Example 1; to 6.8 to 9.9 seconds, inclusive), and permits analysis of more data points. This embodiment demonstrates the assay reagent can be adapted to support a higher number of data points collected, or, as demonstrated in Example 1, a lower number of data points collected and less time required to carry out the data collection.

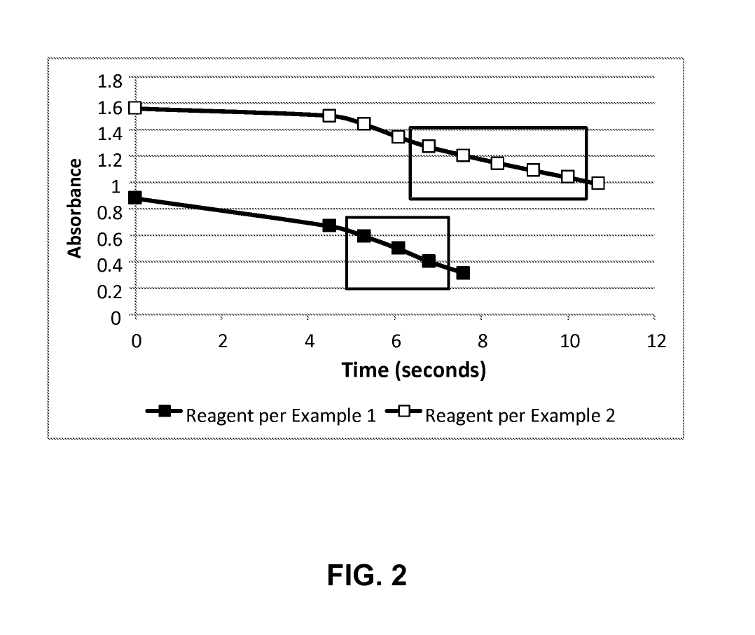

FIG. 2 presents the difference in measurement (absorbance versus time) of a single well for the same dilution of a sample of carbonic anhydrase measured using assay reagent as described in Example 1, compared to that described in Example 2.

When required, such as for stability of certain forms of carbonic anhydrase, additional compounds, such as salts, for example sodium chloride, can be added to the assay reagent. This embodiment of the assay reagent leads to an improved measure of carbonic anhydrase activity for forms of the enzyme which, in the absence of salt, may aggregate, precipitate, or otherwise change their form and alter the result in the assay measurement of CO₂ absorption enhancement. The required concentration of salt is expected to be enzyme dependent, and the assay can be used to determine the optimal salt concentration required for optimal enzyme activity determination.

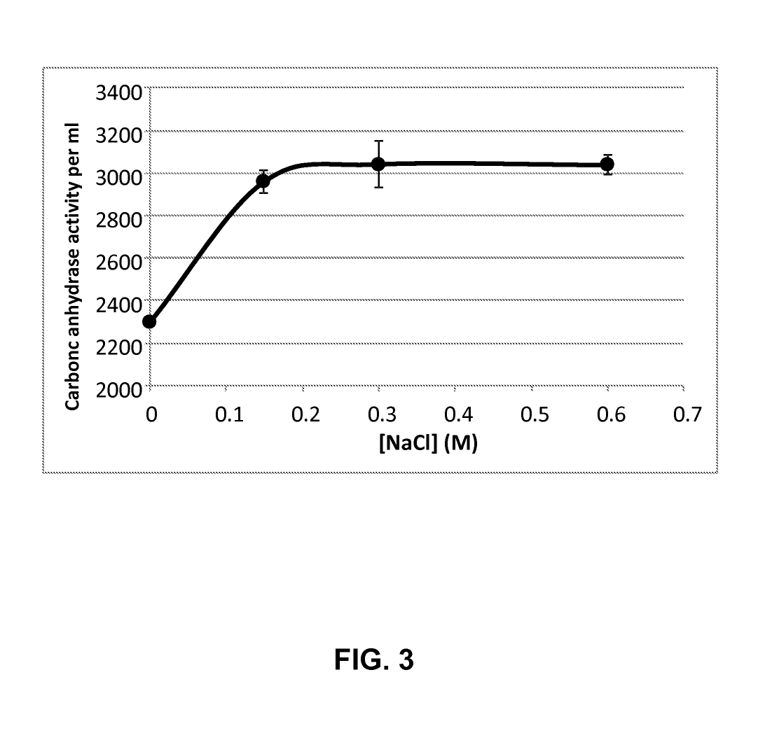

FIG. 3 presents the effect of salt concentration on the determination of carbonic anhydrase activity for a form of the enzyme that precipitates in the absence of salt.

### Example 3

### Identification of Dilution Range for Measurement of CO₂ Absorption Enhancement by Carbonic Anhydrase Using Bicine and Cresol Red

The first time a sample with unknown carbonic anhydrase activity is evaluated for enzyme activity, a pre-test can be used to identify a dilution range for the enzyme wherein the enzyme will be saturated with substrate; and to identify whether the assay reagent alone may be used as the blank or whether a modified enzyme sample should be used. The modification involves removing active enzyme from the sample or inactivating the enzyme in the sample. A 96-well microtiter pre-test plate is prepared containing duplicate wells of each test sample dilution. A broad dilution range is selected for the pre-test plate (for example, one thousand to one-hundred thousand-fold dilutions). Both the unmodified and the modified enzyme samples are identically diluted and 100 microliters plated alongside at least 16 replicate wells containing assay reagent alone. Data is pasted into the Excel Spreadsheet data processing template and analyzed to determine which dilution(s) passed the “linearity test” and T-test, as described in Example 1. These dilutions help inform which 5 or 6×0.7-fold dilutions should be used. Furthermore, a comparison of slopes (change in absorbance per unit time) for assay reagent alone versus modified enzyme sample will inform the selection of the appropriate blank. If the modified enzyme sample (at the same dilution range selected for the enzyme solution) is faster than the assay reagent alone it suggests another component of the enzyme test sample is capable of promoting CO₂ hydration or pH change independent of the enzyme. In this case the modified enzyme sample should be used as the blank, and can be termed a “modified blank” for clarity.

### Example 4

### Measurement of CO₂ Absorption Enhancement by Carbonic Anhydrase Using Tris and Bromothymol Blue

The experiment in this example was carried out as described in Example 1 except that in addition to bicine/cresol red, a second microtiter plate was made that used 25 mM Tris/HCl buffer in place of bicine buffer and 80 micromolar (0.05 g/L) bromothymol blue in place of cresol red. The pKa of Tris is 8.0 and the pKa of bromothymol blue is 7.1, which are dissimilar pKa values, whereas bicine and cresol red have closely matching pKa values of 8.35 and 8.32, respectively. The same enzyme sample was used for both tests, with the same dilution range, and aqueous CO₂ substrate was prepared as described in Example 1. Assay reagent (in this example, the combination of Tris/HCl buffer and bromothymol blue) was used for the blanks. Table 2 shows a comparison of analysis results obtained using the two different buffer/indicator pairs by presenting the average linear ratios for each of three dilutions for both the assay blanks and the assay samples. Analysis using bicine/cresol red (with closely matching pKa values) gives better results (average linear ratio values are all close to the value 1) than Tris/bromothymol blue, with divergent pKa values (average linear ratio values are not near the value of 1). Furthermore, the number of wells passing both the “linearity test” and the T-test was higher for analyses carried out with bicine/cresol red than with Tris/bromothymol blue.

TABLE 2

| | Assay | | | | | |
|---|---|---|---|---|---|---|
| | Tris/Bromothymol | | | | | |
| | blue | | | Bicine/Cresol red | | |
| Dilution set | 1 | 2 | 3 | 1 | 2 | 3 |
| Average linear ratio | | | | | | |
| Assay blank | 0.75 | 0.71 | 0.69 | 1.11 | 1.11 | 1.04 |
| Assay sample | 0.88 | 0.73 | 0.70 | 1.20 | 1.13 | 1.08 |
| Number of wells passing | | | | | | |
| “linearity test” | | | | | | |
| and T-test | | | | | | |
| Assay blank | 1 | 0 | 0 | 7 | 5 | 8 |
| Assay sample | 0 | 0 | 0 | 3 | 6 | 8 |

### Example 5

### Measurement of CO₂ Absorption Enhancement by Carbonic Anhydrase Before and after Heat Treatment Using Bicine and Cresol Red

Carbonic anhydrase temperature tolerance was evaluated by measuring enzyme activity before and after heat treatment according to the method of Example 1. A carbonic anhydrase of microbial origin was incubated in 20 wt % K₂CO₃/KHCO₃ buffer at alkaline pH at 70° C. for 8 days. The data are presented in Table 3. The results show that the enzyme is stable at 70° C. for 48 h, losing activity thereafter.

TABLE 3

| Day | Activity (U/mL) | % activity remaining |
|---|---|---|
| 0 | 920 | 100% |
| 2 | 1060 | 115% |
| 5 | 460 | 50% |
| 8 | 110 | 12% |

### Example 6

### Measurement of CO₂ Absorption Enhancement when a Beneficial Compound is Combined with Carbonic Anhydrase Using Bicine and Cresol Red

Carbonic anhydrase activity improvement was evaluated by measuring enzyme activity before and after zinc addition according to the method of Example 1. A carbonic anhydrase sample of microbial origin known to be deficient in zinc was incubated in either the presence or absence of 3.5 mM ZnSO₄×7 H₂O or ZnCl₂ overnight at room temperature. The data are presented in Table 4. The results show that the addition of zinc led to a dramatic (around 10-fold) activity improvement; and demonstrates the assay's ability to measure the impact of a non-catalytic compound on catalyst performance. Without being limited to a particular mechanistic explanation the result can be explained by the added zinc being incorporated into zinc deficient active sites of the enzyme sample, leading to the observed higher enzyme activity. This example shows the usefulness of the assay in identifying compounds beneficial to catalyst performance.

TABLE 4

| Condition | Activity (U/mL) | fold improvement |
|---|---|---|
| assay blank | 0 | — |
| assay blank + zinc | 0 | — |
| assay sample | 640 | — |
| assay sample + zinc | 6400 (ZnSO₄ × 7H₂O) | 10 (ZnSO₄ × 7H₂O) |
| | 5800 (ZnCl₂) | 9 (ZnCl₂) |

### Example 7

### Composition for Absorption of CO₂ Comprising Carbonic Anhydrase and Bicine

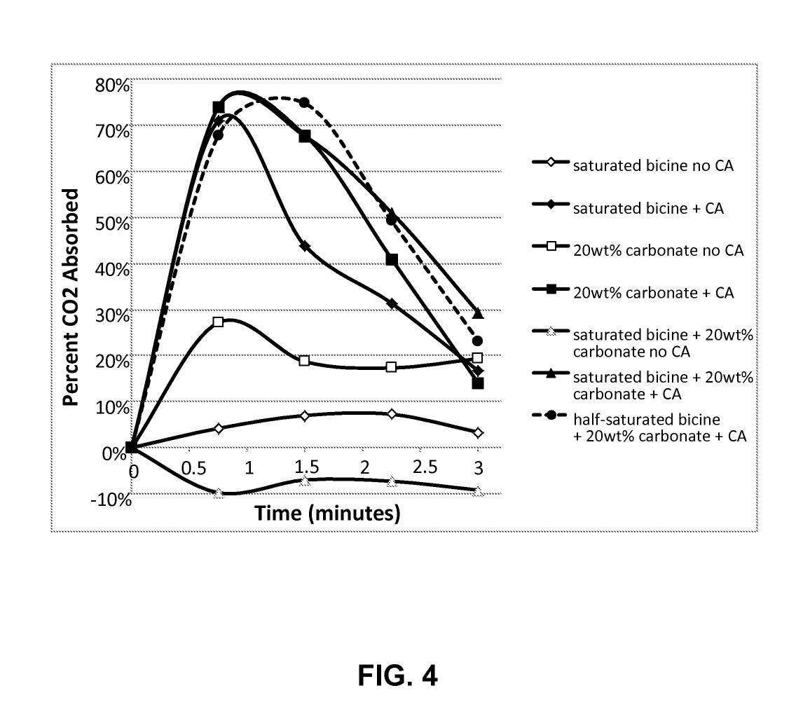

The CO₂ absorption of a solution comprising carbonic anhydrase and bicine was evaluated using a laboratory scale (250 ml) bubble tank reactor supplied with a humidified mixture of nitrogen and CO₂ gas. The mixture was held at a CO₂ concentration of nominally 14% to mimic the CO₂ concentration in flue gas from a coal fired power plant. A total gas flow rate of 2.5 L per minute was employed in order to achieve less than 100% CO₂ absorption and ensure all measurements were recorded within the range of the equipment used. An immersed sparger was used to bubble the gas mixture through the test solvent at room temperature (approximately 20° C.). Antifoam was included in the test solvent. The amount of CO₂ exiting the bubble tank reactor was quantified using gas chromatography. FIG. 4 presents the data, expressed in terms of percent CO₂ absorbed over time.

Test 1: An experiment was performed to compare the CO₂ absorbed in a 1.8 M aqueous bicine solution (pH adjusted to 10 using potassium hydroxide), in either the presence or absence of carbonic anhydrase (1000 activity units, as determined using the assay as described in Example 1). The results demonstrate that bicine is a more effective CO₂ absorption solvent in combination with carbonic anhydrase. The initial rate of CO₂ absorption is higher for the combination of bicine with carbonic anhydrase than for bicine alone. As the experiment proceeds, the total amount of CO₂ absorbed over time is a measure of the CO₂ loading in the solvent. The amount of CO₂ absorbed is higher for the combination of bicine with carbonic anhydrase than for bicine alone.

Test 2: The CO₂ absorption performance of a 1.8 M aqueous bicine solution (pH 10) with or without carbonic anhydrase was compared to a CO₂ loaded 20 wt % potassium carbonate solution (pH 10), prepared by mixing 122 g potassium carbonate with 106 g potassium bicarbonate in 771 g of water to achieve a 1 kg solution. The test with carbonic anhydrase contained an equivalent enzyme dose to Test 1. Results demonstrate that both the initial rate of absorption and the total CO₂ loading in the solvent are higher for potassium carbonate than for bicine under the test conditions.

Test 3: The CO₂ absorption performance of a combination of bicine, carbonic anhydrase, and 20 wt % potassium carbonate equivalent was evaluated. Tests were conducted using both 1.8 M and 0.9 M bicine solutions in combination with 20 wt % potassium carbonate equivalent (pH 10) in either the presence of absence of carbonic anhydrase at equivalent dose to Test 1. Results indicate an improvement to solvent CO₂ loading in solutions comprising carbonate, bicine (at low and high concentrations) and carbonic anhydrase. The solution containing a high bicine concentration resulted in a CO₂ loaded solid/liquid slurry, whereas no solids were observed in any of the other CO₂ loaded solutions. This result shows the combination of bicine, carbonic anhydrase and 20 wt % potassium carbonate equivalent gave the highest CO₂ absorption performance.

Although the foregoing has been described in some detail by way of illustration and example for the purposes of clarity of understanding, it is apparent to those skilled in the art that any equivalent aspect or modification, may be practiced. Therefore, the description and examples should not be construed as limiting the scope of the invention.

The present invention may be further described by the following numbered paragraphs:

[A1] A method for analyzing catalytic activity of a sample comprising conducting an assay for measuring catalytic activity of a sample of a catalyst, wherein an assay liquid used in the assay comprises a sample catalyst, a buffer compound and a pH indicator, wherein the buffer compound is an N,N-disubstituted derivative of an amino acid which comprises a tertiary amine functional group, and the pH indicator has the following characteristic: (i) has a pKa which is similar to the pKa of the buffer compound; and (ii) enables a color transition point which occurs outside of the buffer range controlled by the buffer compound.

[A2] The method of paragraph [A1], wherein the buffer compound is bicine.

[A3] The method of paragraphs [A1] or [A2], wherein the pH indicator is cresol red

[A4] The method of paragraph [A1], wherein the pKa of the buffer compound differs from the pKa of the pH indicator by no more than 0.5 unit, e.g., no more than 0.4 units, no more than 0.3 units, no more than 0.2 units, no more than 0.1 units, no more than 0.09 units, no more than 0.08 units, no more than 0.07 units, no more than 0.06 units, no more than 0.05 units, no more than 0.04 units, or no more than 0.03 units.

[A5] The method of any of paragraphs [A1]-[A4] wherein the sample catalyst is an enzyme.

[A6] The method of any of paragraphs [A1]-[A5], wherein the sample catalyst is one or more carbonic anhydrases.

[A7] The method of any of paragraphs [A1]-[A6], wherein the buffer concentration in the assay liquid is between 1 mM and 2 M.

[A8] The method of any of paragraphs [A1]-[A7], wherein buffer maintains the initial pH above pH 7.5.

[A9] A method for analysis of CO₂ absorption, comprising conducting an assay for measuring catalytic activity of a sample catalyst, wherein an assay liquid used in the assay comprises a sample catalyst comprising one or more carbonic anhydrases, bicine, and cresol red.

[A10] A composition for absorption of CO₂ comprising one or more carbonic anhydrases and a buffer compound comprising N,N-disubstituted derivative of an amino acid which comprises a tertiary amine functional group, wherein the buffer compound is present in an amount effective for providing CO₂ absorption, and wherein the one or more carbonic anhydrases is present in an amount effective for enhancing the rate of CO₂ absorption into an aqueous preparation of the buffer compound.

[A11] The composition of paragraph [A10], wherein the buffer compound is bicine.

[A12] A method for improving the CO₂ absorption rate of an aqueous preparation of a buffer compound comprising an N,N-disubstituted derivative of an amino acid which comprises a tertiary amine functional group, comprising adding one or more carbonic anhydrases to the aqueous preparation.

[A13] The method of paragraph [A12], wherein the buffer compound is bicine.

[A14] A method for improving the activity of a carbonic anhydrate comprising adding zinc (e.g., ZnCl₂ or ZnSO₄) to a composition comprising one or more carbonic anhydrases, wherein the zinc is added in an amount effective to increase the activity of the carbonic anhydrase.

[A15] The method of paragraph [A1], wherein the method is a method for CO₂ absorption or CO₂ desorption.

[A16] A composition comprising one or more carbonic anhydrases and zinc ions (e.g., ZnCl₂ or ZnSO₄), wherein the zinc ions added to the composition independent of the natural zinc content of the one or more carbonic anhydrases, and wherein the zinc ions are added in an amount effective to increase the catalytic activity of a carbonic anhydrase.

[B1] A method for analyzing catalytic activity comprising:
mixing an assay substrate with an assay reagent,
wherein the assay reagent comprises a sample catalyst, a buffer compound, and a pH indicator;
wherein the buffer compound is an N,N-disubstituted derivative of an amino acid which comprises a tertiary amine functional group; and
wherein the pH indicator: (i) has a pKa which is similar to the pKa of the buffer compound; and (ii) enables a color transition point which occurs outside of the buffer range controlled by the buffer compound.

[B2] The method of paragraph [B1], wherein the buffer compound is bicine.

[B3] The method of paragraph [B1] or [B2], wherein after mixing the assay substrate with the assay reagent, the buffer compound is at a concentration between 1 mM and 2 M, e.g., between 5 mM and 1.5 M, between 10 mM and 1 M, or between 10 mM and 100 mM.

[B4] The method of any one of paragraphs [B1]-[B3], wherein the pKa of the buffer compound differs from the pKa of the pH indicator by no more than 0.5 unit, e.g., no more than 0.4 units, no more than 0.3 units, no more than 0.2 units, no more than 0.1 units, no more than 0.09 units, no more than 0.08 units, no more than 0.07 units, no more than 0.06 units, no more than 0.05 units, no more than 0.04 units, or no more than 0.03 units.

[B5] The method of any one of paragraphs [B1] or [B4], wherein the pH indicator is cresol red.

[B6] The method of any one of paragraphs [B1]-[B5], wherein the sample catalyst is one or more enzymes.

[B7] The method of paragraph [B6], wherein the one or more enzymes is one or more carbonic anhydrases.

[B8] The method of any one of paragraphs [B1]-[B7], wherein after mixing the assay substrate with the assay reagent, the total amount of carbonic anhydrase is below 2 g/L assay liquid, e.g., below 1.5 g/L, below 1 g/L, below 0.6 g/L, below 0.3 g/L, below 0.1 g/L, below 0.05 g/L, below 0.01 g/L, below 0.005 g/L, below 0.001 g/L, or below 0.0005 g/L.

[B9] The method of any one of paragraphs [B1]-[B8], wherein pH of the assay reagent is above pH 7.5, e.g., between pH 8 and pH 10, between pH 8 and pH 9, between pH 8 and pH 8.5, or between pH 8.3 and pH 8.7.

[B10] The method of any one of paragraphs [B1]-[B9], wherein the assay reagent further comprises one or more salts (e.g., one or more metal halides, carbonates, bicarbonates, sulfates, phosphates, and/or nitrates, such as potassium carbonate).

[B11] A method for analyzing CO₂ absoption, comprising mixing an assay substrate with an aqueous solution of bicine, cresol red, and one or more carbonic anhydrases.

[B12] The method of paragraph [B11], wherein the bicine concentration of the mixed aqueous solution is between 1 mM and 2 M, e.g., between 5 mM and 1.5 M, between 10 mM and 1 M, or between 10 mM and 100 mM.

[B13] The method of paragraph [B11] or [B12], wherein the total amount of carbonic anhydrase in the mixed aqueous solution is below 2 g/L assay liquid, e.g., below 1.5 g/L, below 1 g/L, below 0.6 g/L, below 0.3 g/L, below 0.1 g/L, below 0.05 g/L, below 0.01 g/L, below 0.005 g/L, below 0.001 g/L, or below 0.0005 g/L.

[B14] The method of any one of paragraphs [B11]-[B13], wherein the pH of the mixed aqueous solution is above pH 7.5, e.g., between pH 8 and pH 10, between pH 8 and pH 9, between pH 8 and pH 8.5, or between pH 8.3 and pH 8.7.

[B15] The method of any one of paragraphs [B11]-[B14], wherein the aqueous solution further comprises one or more salts (e.g., one or more metal halides, carbonates, bicarbonates, sulfates, phosphates, and/or nitrates, such as potassium carbonate).

[B16] A composition comprising (a) one or more carbonic anhydrases and (b) a buffer compound comprising N,N-disubstituted derivative of an amino acid which comprises a tertiary amine functional group,
wherein the buffer compound is present in an amount effective for providing CO₂ absorption, and wherein the one or more carbonic anhydrases is present in an amount effective for enhancing the rate of CO₂ absorption into the composition.

[B17] The composition of paragraph [B16], wherein the buffer compound is bicine.

[B18] The composition of paragraph [B16] or [B17], wherein the composition is an aqueous solution.

[B19] The composition of paragraph [B18], wherein the buffer compound is at a concentration of at least 0.5 M, e.g., at least 1.0 M, such as between 0.5 M and 5 M.

[B20] The composition of paragraph [B18] or [B19], wherein the pH is greater than 8.0, e.g., pH greater than 9.0, or pH greater than 10.0.

[B21] The composition of any one of paragraphs [B16]-[B20], further comprising one or more salts (e.g., one or more metal halides, carbonates, bicarbonates, sulfates, phosphates, and/or nitrates, such as potassium carbonate).

[B22] A method for improving the CO₂ absorption rate of an aqueous solution, wherein the solution comprises an N,N-disubstituted derivative of an amino acid which comprises a tertiary amine functional group, the method comprising mixing one or more carbonic anhydrases to the aqueous solution.

[B23] The method of paragraph [B22], wherein the N,N-disubstituted derivative of an amino acid which comprises a tertiary amine functional group is bicine.

[B24] The method of paragraph [B22] or [B23] wherein the N,N-disubstituted derivative of an amino acid which comprises a tertiary amine functional group is at a concentration in the mixed solution of at least 0.5 M, e.g., at least 1.0 M, such as between 0.5 M and 5 M.

[B25] The method of any one of paragraphs [B22]-[B24], wherein the pH of the mixed CO₂-lean solution is greater than 8.0, e.g., pH greater than 9.0, or pH greater than 10.0.

[B26] The method of any one of paragraphs [B22]-[B25], wherein the mixed solution further comprises one or more salts (e.g., one or more metal halides, carbonates, bicarbonates, sulfates, phosphates, and/or nitrates, such as potassium carbonate).

[B27] The method of paragraph [B22], wherein the N,N-disubstituted derivative of an amino acid which comprises a tertiary amine functional group is bicine, and wherein the mixed solution further comprises one or more carbonate salts.

1. A method for analyzing catalytic activity comprising:
mixing an assay substrate with an assay reagent,
wherein the assay reagent comprises a sample catalyst, a buffer compound, and a pH indicator;
wherein the buffer compound is an N,N-disubstituted derivative of an amino acid which comprises a tertiary amine functional group; and
wherein the pH indicator: (i) has a pKa which is similar to the pKa of the buffer compound; and (ii) enables a color transition point which occurs outside of the buffer range controlled by the buffer compound.

2. The method of claim 1, wherein the buffer compound is bicine.

3. The method of claim 1, wherein after mixing the assay substrate with the assay reagent, the buffer compound is at a concentration between 1 mM and 2 M, e.g., between 5 mM and 1.5 M, between 10 mM and 1 M, or between 10 mM and 100 mM.

4. The method of claim 1, wherein the pKa of the buffer compound differs from the pKa of the pH indicator by no more than 0.5 unit.

5. The method of claim 1, wherein the pH indicator is cresol red.

6. The method of claim 1, wherein the sample catalyst is one or more enzymes.

7. The method of claim 6, wherein the one or more enzymes is one or more carbonic anhydrases.

8. The method of claim 1, wherein after mixing the assay substrate with the assay reagent, the total amount of carbonic anhydrase is below 2 g/L assay liquid.

9. The method of claim 1, wherein pH of the assay reagent is above pH 7.5.

10. The method of claim 1, wherein the assay reagent further comprises one or more salts.

11-21. (canceled)

22. A method for improving the CO₂ absorption rate of an aqueous solution, wherein the solution comprises an N,N-disubstituted derivative of an amino acid which comprises a tertiary amine functional group, the method comprising mixing one or more carbonic anhydrases to the aqueous solution.

23. The method of claim 22, wherein the N,N-disubstituted derivative of an amino acid which comprises a tertiary amine functional group is bicine.

24. The method of claim 22, wherein the N,N-disubstituted derivative of an amino acid which comprises a tertiary amine functional group is at a concentration in the mixed solution of at least 0.5 M.

25. The method of claim 22, wherein the pH of the mixed solution is greater than 8.9.

26. The method of claim 22, wherein the mixed solution further comprises one or more salts.

27. The method of claim 22, wherein the N,N-disubstituted derivative of an amino acid which comprises a tertiary amine functional group is bicine, and wherein the mixed solution further comprises one or more carbonate salts.
